(19) United States
(12) Patent Application Publication
(10) Pub. No.: US 2011/0158961 A1
(43) Pub. Date: Jun. 30, 2011

(54) MATERIALS AND METHODS RELATING TO CELL BASED THERAPIES

(75) Inventor: Paul Shiels, Glasgow (GB)

(73) Assignee: THE UNIVERSITY COURT OF THE UNIVERSITY OF GLASGOW, Glasgow, GB (GB)

(21) Appl. No.: 12/991,878

(22) PCT Filed: May 8, 2009
(86) PCT No.: PCT/GB09/01149

§ 371 Date: Feb. 22, 2011

Related U.S. Application Data
(60) Provisional application No. 61/052,098, filed on May 9, 2008.

(51) Int. Cl. A61K 35/12; C12N 5/00; C12N 5/071; C12N 5/02; A61P 25/28; A61P 25/16; A61P 27/02; A61P 9/00; A61P 3/10

(52) U.S. Cl. 424/93.7; 435/325; 435/366; 435/378

(57) ABSTRACT

The invention relates to the provision of a novel cell population that can be used for tissue regeneration and the treatment of disease states associated with cell degeneration for age related tissue changes. The cell population are derived from adult stem/progenitor cells which are characterised by being positive or negative to the Thy1.1 cell marker.

### CROSS-REFERENCE TO RELATED APPLICATIONS

This application is copending with, shares at least one common inventor with, and claims priority to U.S. provisional patent application No. 61/052,098 filed May 9, 2008. The prior application is hereby incorporated by reference in its entirety.

### FIELD OF THE INVENTION

The present invention relates to the provision of novel cell populations that can be used for tissue regeneration and the treatment of disease states associated with cell degeneration or age related tissue changes. Particularly, but not exclusively, the invention provides materials and methods arising from the determination of novel cell populations that exhibit bipotentiality for differentiation into both pancreatic and hepatic cell types.

### BACKGROUND OF THE INVENTION

Stem cells are unspecialised cells that have the capacity to proliferate for long periods in culture and which can be induced to become specialized cell types. Stem cells can be isolated primarily from the embryo or adult though these appear to have distinct character and function.

Stem cells have been isolated from the embryos (embryonic/embryo stem cells—ESCs) of numerous mammalian species. Those from the mouse have been subject of intense study for the over twenty years and paved the way for the isolation of human ESCs which have been isolated and worked on since 1998.

ESCs are typically derived from the embryonic blastocyst where in vivo they go on to give rise to all subsequent developmental cell types. Adult stem cells (ASCs) on the other hand, are present in numerous tissues where they enable replacement for cells lost through insult and, or, wear and tear. These cells have the potential to provide cellular based therapies for the treatment of diseases such as Diabetes and Parkinson's disease, where there is cell loss and damage leading to the specific pathology. They also have great potential for screening drugs, toxicological investigations, investigating developmental programming, treating age related tissue loss and degeneration. They also have potential for tissue regeneration following surgical removal, insult or as part of cosmetic surgical procedures.

ASCs are undifferentiated cells found among differentiated cells in an adult tissue or organ, they are unspecialised and can self renew themselves maintaining a capacity to differentiate to yield the major cell types of a tissue or organ. ASCs are considered to maintain and effect tissue repair. The origin of adult stem cells in mature tissues is unknown and the degree of their plasticity remains to be determined. Their use in transplantation is widely known. ASCs from bone marrow have been used in transplants for 30 years. The use of adult non-HSCs, their efficacy, plasticity and safety on long term follow up remains unproven. Reports of non stromal ASCs remains debated in the field, though neural stem cells are now established and accepted as a bone fide ASC type.

The proven pluripotent character of ESCs and the ability to grow them in large numbers makes them attractive candidates for a cell based therapy. There is a severe limitation for the use of ASCs in this context, where such cells are considered rare and their growth conditions not sufficiently defined to produce suitable cells in sufficient numbers for potential therapies.

ASCs do have the critical advantage, however, in that they can be derived from ‘self’, hence any patient would receive their own cells and not be required to suffer the deleterious side-effects of immuno-suppression to prevent rejection, including a significantly enhanced risk of cancer. Limited potency/plasticity is also considered to be an enhanced safety factor, in that aberrant cell differentiation would be limited and the risk of neoplasia reduced.

The common developmental origin of the liver and pancreas suggests that these organs may share common stem/progenitor cell populations. There is considerable indirect evidence in support of this hypothesis. Explant experiments have demonstrated that ventral endoderm which expresses Pdx-1 is diverted to a hepatic lineage by proximity to cardiac mesoderm (Deutsch et al., 2001). Cells with hepatocellular properties have also been observed in ductal areas within murine pancreas. Mice fed on a copper-deficient diet acquire pancreatic damage, loss of acinar cells and after 4-6 weeks, hepatic oval-like cells are observed (Rao et al., 1986; Rao and Reddy, 1995). Furthermore, pancreatic cells isolated from rats subjected to a copper deficient diet transplanted to spleen, demonstrated differentiation into hepatocytes, both morphologically and functionally, by integrating into the parenchymal structure and expressing mature liver specific proteins (Dabeva et al., 1997). Suspensions of wild-type mouse pancreatic cells transplanted into syngenic recipients deficient in fumarylacetoacetate hydrolase, with subsequent tyrosinemia, have also been reported to result in biochemical rescue by donor-derived cells with normalisation of liver function (Wang et al., 2001). Furthermore, transgenic overexpression of KGF in the adult mouse pancreas results in the occurrence of hepatocytes within the pancreatic islets and concomitant pancreatic duct proliferation (Krakowski et al., 1999b; Krakowski et al., 1999a).

Several putative pancreatic progenitor cells have been characterised from both pancreatic ductal and islet cells (Abraham et al., 2004; Cornelius et al., 1997; Lechner et al., 2002; Ramiya et al., 2000). One of these cell populations has been observed, after differentiation in vitro, to express alpha feto protein and c-Met, proteins typically expressed by liver cells (Zulewski et al., 2001). More recently, a mesenchymal stem cell population isolated from human pancreatic ductal epithelium has been reported to have the potential for pancreatic, hepatic and mesodermal differentiation (Seeberger et al., 2006). Although these cells could be induced to express Gata 4, albumin and TAT (tyrosine amino transferase), no functional assessment has been reported.

The present inventor previously determined a further cell type, Pancreatic Derived Pathfinder Cells (PDPCs), isolated from adult rat pancreatic ducts which show multipotency and functional efficacy in an STZ diabetes model (Shiels 2004; and WO 2006/120476, incorporated herein by reference). In adult rat liver, a population of putative stem cells can be induced after induction by chemical injury, or partial hepatectomy (Petersen et al., 1998; Petersen et al., 2003).

### SUMMARY OF THE INVENTION

There is a continuing need to provide cell populations which can be used in cell based therapies to treat diseases particularly associated with cell degeneration. Diabetes is an example of such a disease. Insulin in the pancreas is made by insulin secreting beta cells. In vivo, beta cell turnover is thought to take place throughout life, though controversy exists as to the origin of the replacement cells. Diabetes is one example therefore of a disease where provision of cells (particularly cells derived from self) analogous to fully functioning native cells provides an important alternative to treatment. In the case of diabetes, the provision of cells analogous to beta cells in that they can produce insulin, offers a significant cell-based therapy for the disease.

With this and other diseases in mind, the inventor has isolated subpopulations of cells derived from the stem/progenitor cell population from adult pancreatic ducts.

In some embodiments, a sub-population of cells provided herein is Pdx-1 (HUMAN: NP_{—}000200.1 GI:4557673; RAT: NP_{—}074043.3 GI:50838802) positive. The presence of Pdx-1 expression indicates that cells have potential as a source of non beta cell derived insulin.

In some embodiments, a subpopulation of adult stem/progenitor cells provided herein is characterized by the presence or absence of Thy1.1 (CD90) (HUMAN: NP_{—}006279). Properties of Thy1.1 positive and negative subpopulations are discussed below.

### 1. Thy1.1 Positive Cells

In maintenance (non-differentiation) media, a Thy1.1 positive subpopulation of cells is Pdx-1 positive, insulin negative and glucagon negative. A Thy1.1 positive cell population, when placed in pancreatic differentiation media, initially shows fibroblast-like morphology and then forms matted cell clusters which eventually detach from the parent cell layer. Resulting differentiated cell clusters are positive for Pdx-1, insulin and glucagon (FIG. 4 panel B).

A surprising determination in relation to Thy1.1 positive subpopulations of cells described herein is that they are at least bipotent. Specifically, when provided with the appropriate differentiation media, Thy1.1 positive cells are able to differentiate into either pancreatic or hepatic cell types.

### 2. Thy1.1 Negative Cells

In a non-differentiated state, a Thy1.1 negative cell population is positive for Pdx-1, but negative for insulin and glucagon. When grown in differentiation media, Thy1.1 negative cells showed no morphological changes, but insulin transcription was detected. Accordingly, a Thy1.1 negative population provides a novel source of non-beta cell derived insulin.

Accordingly, at its most general, the present invention provides materials and methods for treating diseases and conditions of ageing based on a cell based therapy using a novel sub-population of cells derived from the multipotent adult stem cell population, the sub-population being Pdx-1 positive.

Such a cell population provides potential for cell based therapy of diseases such as Diabetes and neurodegenerative disorders such as Parkinson's disease.

An adult stem cell population (also known as progenitor cells) can be derived from adult pancreatic tissue, e.g., human, rat, mouse, primates, pig etc. The adult tissue is preferably pancreatic tissue, but may also be tissue derived from other organs such as breast, bone marrow, heart, liver or kidney.

In one embodiment of the invention, adult stem cells are derived from adult rat pancreas which have been deposited in accordance with the Budapest Treaty 1977 at The European Collection of Cell Cultures, Porton Down, Salisbury Wiltshire, UK, SP4 0JG on 12 May 2005 by The University Court of the University of Glasgow under ECACC No. Q6203. These cells are hereinafter known as PDPCs (pancreas derived progenitor cells).

As mentioned above, the inventor has also determined two further subpopulations with reference to the marker Thy1.1 (CD90) (HUMAN: NP_{—}006279). These two sub-populations have distinct but equally important properties. In particular, Thy1.1 positive cells exhibit bipotentiality for differentiation into both pancreatic and hepatic cell types. In some embodiments, a cell subpopulation described herein provides a non-beta cell source of insulin. In some embodiments, a cell subpopulation described herein provides a cell type to evaluate toxicity of a test substance (e.g., for toxicological testing).

Accordingly, in a first aspect, there is provided a population of stem/progenitor cells originating from adult tissue, wherein said cells are capable of growth in a matrigel free culture system in the presence of serum, and wherein the cells are Thy1.1 positive. In some embodiments, Thy1.1 positive cells are also Pdx-1 positive. In some embodiments, Thy1.1 positive cells are Nestin (RAT:NP_{—}037119.1 GI:6981262; HUMAN: NP_{—}006608.1 GI:38176300) positive.

In some embodiments, the percentage of Nestin positive cells in the population is less than 50% as determined by flow cyometry analysis (e.g., less than 40%, 30%, 20%, 10%, 5%, 2%, or 1%). In some embodiments, cells are distinguishable as Nestin positive cells by expression of Nestin nucleic acids, e.g., by PCR.

A cell population may be considered as Thy1.1 positive (CD90 positive) (RAT:LOCUS:P01830 GI135832, NP_{—}006279 GI:19923362) where the percentage of Thy1.1 positive cells is greater than 50%, preferably greater than 60%, more preferably greater than 70%, 80%, 90%, or 95%. In some embodiments, a Thy1.1 positive cell population has greater than 98% purity. The percentage of Thy1.1 positive cells may be determined, e.g., by flow cytometry or by PCR.

In some embodiments, a Thy1.1 positive cell population in accordance with the present invention is positive for expression of one or more of Pdx-1, CD49f, CD147, CD44, c-Met, and Nestin. In some embodiments, a Thy1.1 positive cell population is negative for expression of one or more of CD24, CD45, CD31, c-kit, and CK19. In some embodiments, a Thy1.1 positive cell population in accordance with the present invention has the following cell surface marker profile:

| | |
|---|---|
| Thy1.1 (CD90) | Positive |
| Pdx-1 | Positive |
| CD49f | ~95%+ |
| CD24 | Negative |
| CD147 | ~90%+ |
| CD45 | Negative |
| CD44 | ~85%+ |
| CD71 | low |
| CD31 | Negative |
| C-KIT | Negative |
| CK19 | Negative |
| c-Met | Positive |
| Nestin | Positive |

(Low = approximately less or equal to 5% of cells express the marker)

The invention also provides a pharmaceutical composition comprising said cell population in accordance with this aspect of the invention along with a pharmaceutically acceptable carrier.

In a second aspect of the invention, there is provided a method of producing an isolated bipotent stem cell population from adult mammalian tissue, said method comprising: culturing said adult mammalian tissue; obtaining emergent cell population monolayer; and isolating a subpopulation comprising cells positive for Thy1.1. In some embodiments, at least 50%, 60%, 70%, 80%, 90%, 95% of the subpopulation is Thy1.1 positive.

The method may also include isolating those cells which are positive for Thy1.1 in combination with one or more other cell surface markers provided in the profile provided above (e.g., positive for one or more of Pdx-1, CD49f, CD147, CD44, c-Met, and Nestin and/or negative for one or more of CD24, CD45, CD31, c-kit, and CK19). In some embodiments, the adult mammalian tissue is pancreatic, e.g., derived from pancreatic ducts. In some embodiments, the adult mammalian tissue is breast, liver or kidney. In some embodiments, the adult mammalian tissue is human tissue.

Instead of obtaining adult mammalian tissue in order to obtain emergent cell population monolayer, a method may involve obtaining adult stem cells already isolated, e.g. those deposited at ECACC under accession number Q6203 on 12 May 2005.

In a third aspect, there is provided a method of producing a population of hepatic cells in culture, said method comprising culturing a Thy1.1 positive cell population in accordance with the present invention in medium suitable for hepatic lineage differentiation. As an example (others will be known to those skilled in the art), the culture medium may be a serum-free FGF-4 containing differentiation media.

In a fourth aspect, there is provided a method of producing a population of pancreatic cells in culture, said method comprising culturing a Thy1.1 positive cell population in accordance with the invention in medium suitable for pancreatic lineage differentiation.

The present invention extends to cells and cell populations obtained or obtainable from the method described herein.

In a fifth aspect of the present invention, there is provided a method of treating a disease state associated with cell degeneration or age related tissue change, said method comprising the steps of administering a Thy1.1 positive adult stem cell population according to the invention or a pharmaceutical composition comprising said Thy1.1 positive cell population, to a patient having said disease or age related condition.

A Thy1.1 positive cell population can be administered intravenously or can be transplanted to a disease site.

In some embodiments, the disease is associated with degeneration of pancreatic cells, neuronal cell, cardiovascular cells (e.g. cardiomyocytes), epithelial cells, liver cells or kidney cells.

The disease state to be treated may include diabetes (type I and II), liver disease, kidney disease, eye disease, Parkinson's disease and cardiovascular disease and age related degenerative conditions of the organs and tissues of the body. This aspect of the invention may also be used as a form of cosmetic surgery, e.g. cell regeneration for tissues and to prevent forms of ageing.

In some embodiments, the donor of the cells and the recipient are the same species (e.g., both are human). In some embodiments, the donor of the cells and the recipient are of different species. Accordingly, an embodiment of the present invention includes the use of adult stem cell populations derived from rat in the treatment of human patients.

In a sixth aspect of the invention, there is provided a method of producing a specified differentiated cell population, e.g., pancreatic cells or hepatic cells, said method comprising the steps of providing an adult stem cell population; selecting a cell sub-population using Pdx-1 and/or Thy1.1 markers and optionally one or more other markers identified herein in relation to the Thy1.1 subpopulation cell surface marker profile; and culturing said sub-population of cells in under conditions conducive to cell differentiation.

The invention further provides a cell population in accordance with the first aspect of the invention for use in a method of medical treatment including cosmetic surgery. The method may be to treat a disease state or condition of ageing associated with cell loss or degeneration, e.g., diabetes or Parkinson's disease.

In a sixth aspect of the present invention there is provided a population of cells originating from adult tissue, wherein said cells are capable of growth in a matrigel free culture system in the presence of serum, and wherein the cells are Thy1.1 negative.

In some embodiments, said cell population is Pdx-1 positive. The cell population may also be Nestin positive.

The present invention also provides a pharmaceutical composition comprising an adult stem Thy1.1 negative cell population along with a pharmaceutical acceptable carrier.

In a seventh aspect of the invention there is provided a method of producing an isolated stem cell population from adult mammalian tissue, said method comprising: culturing said adult mammalian tissue; obtaining emergent cell population monolayer; and isolating a subpopulation of cells negative for Thy1.1. In one embodiment, the method further comprises isolating a subpopulation of cells which is also Pdx-1 positive and/or Nestin positive.

In one embodiment, a subpopulation of Thy1.1 negative cells is positive for expression of one or more of CD49f, CD24, CD147, CD44, c-Met, and/or negative for expression of one or more of CD31, c-kit, and ck7. In one embodiment, a subpopulation of Thy1.1 negative cells is isolated which the following cell surface marker profile:

| | |
|---|---|
| Thy1.1 (CD90) | Negative |
| Pdx-1 | Positive |
| CD49f | ~95%+ |
| CD24 | ~80%+ |
| CD147 | ~80%+ |
| CD45 | Negative |
| CD44 | ~60%+ |
| CD71 | low |
| CD31 | Negative |
| c-KIT | Negative |
| ck7 | Negative |
| CK19 | Weak Positive |
| c-Met | Positive |

(Low = approximately less or equal to 5% of cells express the marker)

Instead of obtaining adult mammalian tissue in order to obtain emergent cell population monolayer, the method may involve obtaining adult stem/progenitor cells already isolated, e.g., those deposited at ECACC under accession number Q6203 on 12 May 2005.

In an eighth aspect of the invention there is provide a method of treating diabetes or a disease state associated with reduction in insulin production, said method comprising administering a Thy1.1 negative cell population according to the invention or a pharmaceutical composition comprising said Thy1.1 negative cell population, to a patient having said disease or age related condition.

A Thy1.1 negative cell population can be administered intravenously or it may be transplanted to the disease site.

In some embodiments, the donor of the cells and the recipient are the same species, e.g., human. In some embodiments, the cells and the recipient are of different species. Accordingly, an embodiment of the present invention includes the use of adult rat stem/progenitor cells in the treatment of human patients.

In a ninth aspect of the invention, there is provided a method of producing a specified differentiated cell population that is capable of producing insulin, said method comprising the steps of providing an adult stem cell population; selecting an adult stem cell subpopulation that is positive for Pdx-1 and negative for Thy1.1 markers; and culturing said subpopulation of cells in under conditions conducive to cell differentiation. The method may further include selecting the adult stem cell population on the basis of one or more further markers identified above as part of the Thy1.1 cell surface marker profile.

The invention further provides an adult stem cell Thy1.1 negative cell population in accordance with the present invention for use in a method of medical treatment. In particular, the method may be to treat diabetes.

Also provided herein are methods of producing insulin. A method of producing insulin can include culturing a population of Thy1.1 negative, Pdx-1 positive cells described herein (e.g., a population of Thy1.1 negative, Pdx-1 positive cells derived from adult tissue, e.g., adult pancreatic tissue) under conditions in which insulin is produced. The method can further include isolating insulin from the culture. In some embodiments, the Thy1.1 negative cells are positive for expression of one or more of CD49f, CD24, CD147, CD44, c-Met, and/or negative for expression of one or more of CD31, c-kit, and ck7.

In another embodiment, a method of producing insulin includes culturing a population of Thy1.1 positive, Pdx-1 positive cells described herein (e.g., a population of differentiated Thy1.1 positive, Pdx-1 positive cells derived from adult tissue, e.g., adult pancreatic tissue) under conditions in which insulin is produced. The method can further include isolating insulin from the culture. In some embodiments, a Thy1.1 positive cell population is positive for expression of one or more of Pdx-1, CD49f, CD147, CD44, c-Met, and Nestin and/or negative for expression of one or more of CD24, CD45, CD31, c-kit, and CK19.

Aspects and embodiments of the present invention will now be illustrated, by way of example, with reference to the accompanying figures. Further aspects and embodiments will be apparent to those skilled in the art. All documents mentioned in this text are incorporated herein by reference.

### DESCRIPTION OF THE DRAWINGS

FIG. 1. Morphology in vitro undifferentiated Thy1.1 positive and Thy1.1 negative populations. Hepatic and pancreatic differentiation in vitro of Thy1.1 positive and Thy1.1 negative PDPC populations. (FIG. 1A-C): Little morphological change was observed in Thy1.1 negative populations during differentiation. (FIG. 1A) Undifferentiated Thy1.1 negative PDPCs (FIG. 1B) Day 28 post induction of pancreatic differentiation Thy1.1 negative PDPCs. (FIG. 1C) Day 28 post induction of hepatic differentiation Thy1.1 negative PDPCs. (FIG. 1D-I): Significant morphological changes were observed on induction of Hepatic differentiation of Thy1.1 positive PDPCs. (FIG. 1D) Undifferentiated Thy1.1 positive PDPCs (Fibroblastoid morphology). (FIG. 1E) Day 14 post induction of hepatic differentiation of Thy1 positive PDPCs (FIG. 1F) Day 28 post induction of hepatic differentiation of Thy1.1 positive PDPCs—epithelial morphology predominated with lumenal structures present. (FIG. 1G) Cuboidal morphology of thy1 positive PDPCs day 28 post hepatic induction. (FIG. 1H) Day 14 post induction of pancreatic differentiation of Thy1.1 positive PDPCs. (FIG. 1I) Day 28 post induction of pancreatic differentiation of Thy1.1 positive PDPCs—Islet-like clusters are formed and subsequently detach into media.

FIG. 2. Cell surface characterisation of MACS sorted PDPCs by flow cytometry. (FIG. 2A) Thy1 (CD90) positive populations are negative for the expression of CD24, CD31. CD45, c-kit and low for CD71, but positive for the expression of CD147, CD44 and CD49f. (FIG. 2B) Thy1 (CD90) negative populations are negative for the expression of CD31. CD45, c-kit and low for CD71, but positive for the expression of CD24, CD147, CD44 and CD49f.

FIG. 3. Immunocytochemistry Thy1.1 sorted PDPC populations. Positive controls are shown for albumin (FIG. 3A), Cytokeratin 7 (FIG. 3E), Vimentin (FIG. 3I) and Cytokeratin 19 (FIG. 3M). The undifferentiated Thy1.1 negative PDPC population demonstrate negative staining for albumin (FIG. 3B), Cytokeratin 7 (FIG. 3F), Vimentin (FIG. 3J) but weak staining for Cytokeratin 19 (FIG. 3N). The undifferentiated Thy1.1 positive population were negative for albumin (FIG. 3C), Cytokeratin 7 (FIG. 3G), Vimentin (FIG. 3K) and Cytokeratin 19 (FIG. 3O). However by day 14 in hepatic differentiation media Thy1.1 positive PDPC were positive for albumin (FIG. 3D), and in the lumenal like areas positive for vimentin (FIG. 3L) and Cytokeratin 19 (FIG. 3P) but Cytokeratin 7 remained negative throughout (FIG. 3H)

FIG. 4. RT-PCR of Thy1.1 positive PDPCs (FIG. 4A) and Thy1.1 negative PDPCs (FIG. 4B) in pancreatic differentiation medium. Thy1.1 negative and positive PDPCs were cultured in pancreatic differentiation medium for 28 days. Column: (1) positive control (2) undifferentiation PDPCs, (3) Day 28 differentiated PDPCs, (4-6) No RT controls of samples 1-3.

FIG. 5. RT-PCR of Thy1.1 positive PDPCs and Thy1.1 negative PDPCs in hepatic differentiation media. Thy1.1 positive (FIG. 5A) and negative (FIG. 5B) PDPCs were cultured in hepatic differentiation medium for 28 days. Column: (1) positive control, (2) undifferentiated PDPCs, (3) Day 7, (4) Day 14, (5) Day 21, (6) Day 28, (7-13) No RT controls of samples 1-6. Expression of albumin, CK19 and HNF1alpha was induced in the Thy1.1 positive population but was not observed in the Thy1.1 negative population.

FIG. 6. Undifferentiated Thy1.1 positive PDPC (FIG. 6B) or Thy1.1 negative PDPC populations (FIG. 6C) do not store glycogen. Thy1.1 positive PDPCs after culture in FGF-4 containing media produce and store glycogen (FIGS. 6D and E). Glycogen storage is seen as accumulation of magenta staining when stained by Periodic acid—Schiff. Thy1.1 negative PDPCs after culture in FGF-4 containing hepatic differentiation media do not demonstrate staining (FIG. 6F) Positive control (FIG. 6A).

### DETAILED DESCRIPTION

### Materials and Methods

### Isolation and Maintenance Culture of Rat PDPCs

Pancreatic ducts were isolated from 12 month old Albino Swiss (Glasgow) rats by dissection and minced, prior to seeding in CMRL medium. The PDP cells emerged as a confluent monolayer after approximately 5 weeks in culture. These were then harvested and washed in PBS. PDPCs were maintained in culture in 20 mls CMRL 1066 medium (Invitrogen, Paisley, UK.) supplemented with 10% Foetal Bovine Serum (Sigma, Poole, UK), 2 mM glutamax, 1.25 μg/ml amphotericin B, and 100 u/ml penicillin, 100 μg/ml streptomycin, (all Invitrogen, Paisley, UK) in T75 culture flasks with 0.2 μm filter caps (Corning, UK) at 37° C. in a 5% CO₂ atmosphere. Sub-confluent cultures were passaged by the total removal of culture medium by pipette and the washing of the adherent cells by the addition of 10 mls calcium and magnesium-free Hanks Balanced Salt Solution (HBSS), (Cambrex Bio-Science, Wokingham, UK) to the flask for 5 minutes at room temperature. After the removal of the HBSS from the flask by pipette, 2 mls of Trypsin-Versene solution (200 mg/L Versene, 500 mg/L Trypsin) was added to the flask. The flask was periodically examined microscopically until dissociation of the cell monolayer can be confirmed. Cells were then removed by pipette and re-cultured as above at a density of 1/5 to 1/10 as desired, by the addition of 20 mls of fresh culture medium.

PDPCs were maintained longterm in CMRL 1066 medium (Invitrogen, Paisley, UK) supplemented with 5% Foetal Bovine Serum (Sigma, Poole, UK), 2 mM Glutamax, 1.25 ug/ml Amphotericin B, and 100 u/ml Penicillin/Streptomycin (all Invitrogen, Paisley) in T75 with 0.2 um filter caps at 37° C. in a 5% CO₂ atmosphere. PDPCs grow in a 37° C., humidified 5% CO₂ atmosphere as a monolayer, and were passaged when 90% confluent with trypsin-EDTA (Invitrogen). Cells were counted and replated at a density of 3300 cells/cm².

### Magnetic Activated Cell Sorting

Magnetic activated cell sorting (MACS) was performed for isolation and depletion using 1 ug of primary antibody, mouse anti-rat Thy1.1 (CD90) (Serotec) per 10⁶ target cells for 20 minutes at 4° C. as per the manufacturers protocol (Dynabeads Goat anti mouse IgG (Dynal Biotech)). Sorted cell populations were resuspended cells in maintenance culture media and replated in tissue culture flasks. MACS was performed on each positive and negative sorted population twice before use in experiments. All sorted population were checked with fluorescence activated flow cytometry before use in subsequent differentiation experiments.

### Flow Cytometry Assessment of Cell Surface Antigens

Cells were resuspended in 0.5% BSA in HBSS. They are then centrifuged at 1000×rpm for 10 minutes and the resulting cell pellet is resuspended in HBBS. After a viability count with Trypan blue (Invitrogen, Paisley, UK), 1×10⁶ cells/ml were labelled with 100 μl primary antibody. Primary antibodies used were against CD90 (Serotec MCA47R, 1:75), CD44 (Serotec MCA643, 1:10), CD49f (Serotec MCA2034, 1:50), CD147 (Serotec MCA729, 1:10), c-KIT (Santa Cruz SC-19983, 1:20), CD71 (Serotec MCA 155FT, 1:10), CD24 (BD Biosciences 551133, 1:50), CD45 (BD Biosciences 554875, 1:50), CD31 (Serotec MCA1334GA, 1:50) and CD34 (Santa Cruz sc-7324, 1:50). Secondary antibodies were added in 0.2% BSA/PBS for 45 minutes at 4° C. in the dark. The cells were then washed and centrifuged three times at 1000×rpm in 0.2% BSA/PBS before labelling by the addition of 100 μl FITC-conjugated Fab2 fragment of Rabbit anti-Mouse Immunoglobulins (Dako Cytomation, Ely, UK 1:20) in 0.2% BSA for 45 minutes at 4° C. in the dark. An isotype FITC control was also performed. After washing 3× and centrifuging as before, the resulting cell pellet was resuspended in 1 ml of HBSS and the cells analysed using a Beckman Coulter XL Flow Cytometer (Beckman Coulter, High Wycombe, UK).

### Differentiation Experiments

For pancreatic differentiation Thy1.1 positive and Thy1.1 negative cell populations (Passage 30) were plated at 6600 cells/cm² cell density. After 24 hours maintenance media was removed and monolayers were washed thrice with HBSS. Cells were subsequently cultured in DMEM:F12 (Lonza) supplemented with 1×ITS, 1.25 μg/ml Amphotericin B, and 100 μml Penicillin/Streptomycin (all Invitrogen, UK), Nicotinamide 10 mM (Sigma), KGF 10 ng/ml (Sigma) and 0.2% BSA (Sigma)

For hepatogenic differentiation cells were plated at 6600 cells/cm² in T75 and 6 well plates, and at 2500 cells/cm² in chamber slides (Nunc) at 24 hours maintenance was replaced, after washing thrice with HBSS, DMEM:F12 (Lonza) supplemented with Fibroblast Growth Factor-4 10 ng/ml (Sigma), 1×ITS, 100 μml Penicillin/Streptomycin (Invitrogen) and 0.2% Bovine serum albumin (Sigma). Medium changes were performed thrice weekly and cells were harvested for RNA extraction from undifferentiated Thy1.1 positive and Thy1.1 negative cells at day 0 and day 28 for pancreatic differentiation and at Day 0, 7, 14, 21 and 28 for hepatic differentiation. Cells undergoing hepatic differentiation in chamber slide were washed twice with PBS and fixed with 4% paraformaldehyde for 15 minutes at room temperature between days 10-14. Undifferentiated Thy1.1 positive and negative populations were also grown in chamber slides concurrently and were fixed as above at 90% confluency.

### Immunofluorescence

For staining of intracellular proteins cells were fixed as above. Cells were thrice washed in PBS and permeabilized with 0.1% Triton X-100 (Sigma-Aldrich) for 10 minutes. Slides were incubated with donkey serum for twenty minutes and the incubated with previously optimised primary antibodies diluted in 0.5% BSA/PBS against rat albumin (Abeam ab14255, 1:100), CK19 (Biodesign Int. M08029M, 1:100), CK7 (Chemicon MAB3226, 1:100), CK 18 (Sigma F-4772) and Vimentin (Abeam ab8979, 1:50) for 1 hour. Slides were washed thrice in PBS followed by the appropriate FITC labelled secondary antibody (Abeam ab6749 or Dako Cytomation F0313, Ely, UK). Omission of the primary antibody was performed as negative control. Frozen sections of rat liver were used as positive control. Slides were washed three times before mounting in Vectashield and were visualised and photographed by fluorescence microscopy.

### RT-PCR

Total RNA was extracted by using Trizol® according to the manufacturer's instructions and quantified by GeneQuant analyser. Samples were DNAse treated (Ambion) and reverse transcription to cDNA performed using SuperScript II reverse transcriptase (Invitrogen) according to manufacturer's instructions. No RT negative controls were performed for all samples. RT-PCR was performed using Taq Polymerase (Invitrogen). The housekeeping gene Bactin was used to assess template quality. All PCR reactions were performed using a Peltier Thermal Cycler-200. Nested PCR was performed for pancreatic differentiation experiments.

The following specific olignucleotide primers were used PDX 1, Insulin II and Glucagon (PDX-1 forward, 5-cggccacacagctctacaagg-3 (SEQ ID NO:1), reverse, 5-ctccggttctgctgcgtatgc-3 (SEQ ID NO:2), nested reverse 5-ttccaggcccccagtctcgg-3 (SEQ ID NO:3) (305 bp), Insulin, forward 5-atggccctgtggatccgctt-3 (SEQ ID NO:4); reverse, 5-tgccaaggtctgaaggtcac-3 (SEQ ID NO:5); nested forward, 5-cctgctcatcctctgggagcc-3 (SEQ ID NO:6) (209 bp); Glucagon, forward, 5-gaccgtttacgtggctgg-3 (SEQ ID NO:7); reverse, 5-cggttcctcttggtgttcatcaag-3 (SEQ ID NO:8); nested forward, 5-acaaggcagctggcagcatgc-3 (SEQ ID NO:9) (210 bp). Rat Pancreatic total RNA was reverse transcribed and used as positive control. The following specific oligonucleotide primers were used for hepatic differentiation experiments, albumin (141 bp) forward 5-ctgggagtgtgcagatatcagagt-3 (SEQ ID NO:10), reverse 5-gagaaggtcaccaagtgctgtagt-3 (SEQ ID NO:11), HNF3 beta (63 bp) forward 5-cctactcgtacatctcgctcatca-3 (SEQ ID NO:12), reverse-cgctcagcgtcagcatctt (SEQ ID NO:13), HNF1 (138 bp) alpha forward 5-agctgetcctccatcatcaga-3 (SEQ ID NO:14), reverse 5-tgttccaagcattaagttttctattctaa-3 (SEQ ID NO:15), Gata4 (173 bp) forward 5-catgcttgcagttgtgctag-3 (SEQ ID NO:16), reverse 5-attctctgctacggccagta-3 (SEQ ID NO:17), Alpha-Fetoprotein (124 bp) forward 5-gtcctttcttcctcctggagat-3 (SEQ ID NO:18), reverse 5-ctgtcactgctgatttctctgg-3 (SEQ ID NO:19), CYP2B1(549 bp) forward 5-gagttcttctctgggttcctg-3 (SEQ ID NO:20), reverse 5-actgtgggtcatggagagct-3 (SEQ ID NO:21), CK19 (193 bp) forward 5-agtaacgtgcgtgctgacac-3 (SEQ ID NO:22), reverse 5-agtcgcactggtagcaaggt-3 (SEQ ID NO:23), CK18 (70 bp) forward 5ggacctcagcaagatcatggc-3 (SEQ ID NO:24), reverse 5 ccacgatcttacgggtagttg-3 (SEQ ID NO:25). The PCR products then underwent agarose gel electrophoresis and were visualised by ethidium bromide staining. Rat liver tissue was used as positive control.

### Periodic Acid Schiff Staining

Periodic acid Schiff staining for glycogen storage was performed on undifferentiated Thy1.1 positive and Thy1.1 negative cells and on Thy1.1 positive and negative populations at Day 21 of Hepatic differentiation. Human liver sections were used as positive control. Cells were fixed in 4% paraformaldehyde at room temperature for 10 minutes. Cells were thrice washed in PBS and permeabilized with 0.1% Triton X-100 (Sigma-Aldrich) for 10 minutes and washed with PBS×2 and ddH20×1. Cells were immersed in Periodic acid solution (1 g/dL) for 5 minutes at room temperature. Wells rinsed in distilled water three times. Cells immersed in Schiff's Reagent for 15 mins at room temperature. Cells washed in running tap water for 5 minutes. Cells counterstained in haematoxylin solutions for 90 seconds. Rinse cells in running tap water for 15-30 seconds.

### Results

### Characterisation of Thy1.1 Positive and Negative Populations.

MACs sorting of PDPCs was used to isolate populations expressing Thy1.1 positive cells at more than 98.5% and Thy1.1 negative cells at 98.7% purity respectively. These populations were then cultured and reassessed by flow cytometry regularly every 10-12 days and prior to any differentiation or characterisation experiments.

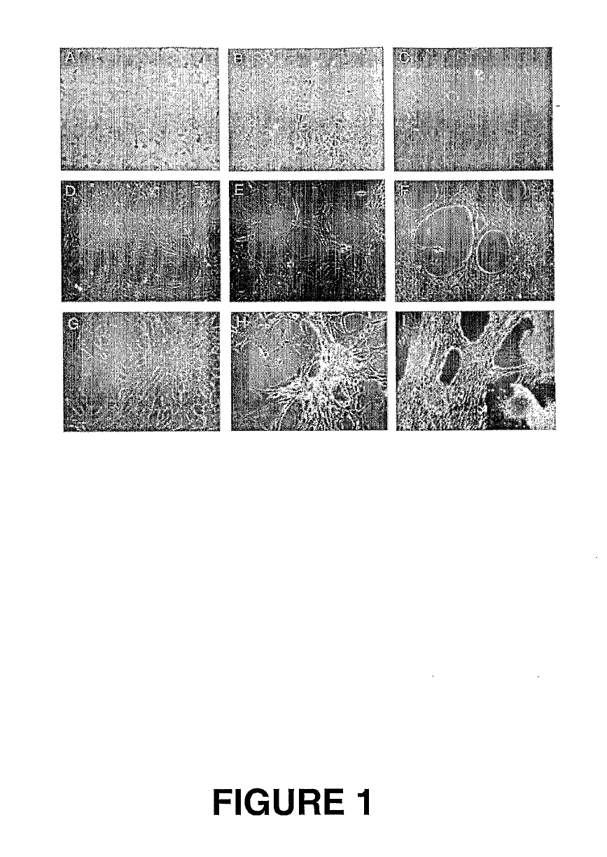

Phenotypically, Thy1.1 positive and negative populations demonstrated distinct differences in morphology: the Thy1.1 positive population exhibited a fibroblast like morphology (FIG. 1, Panel A), while Thy1.1 negative populations exhibited a more epithelial like morphology (FIG. 1, Panel D).

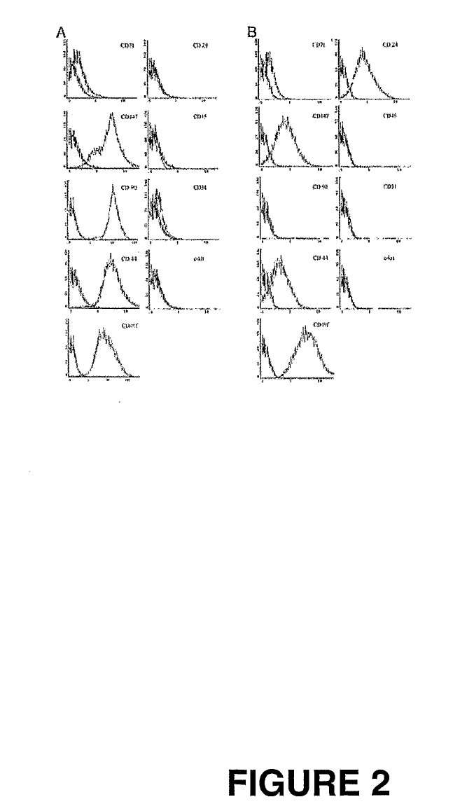

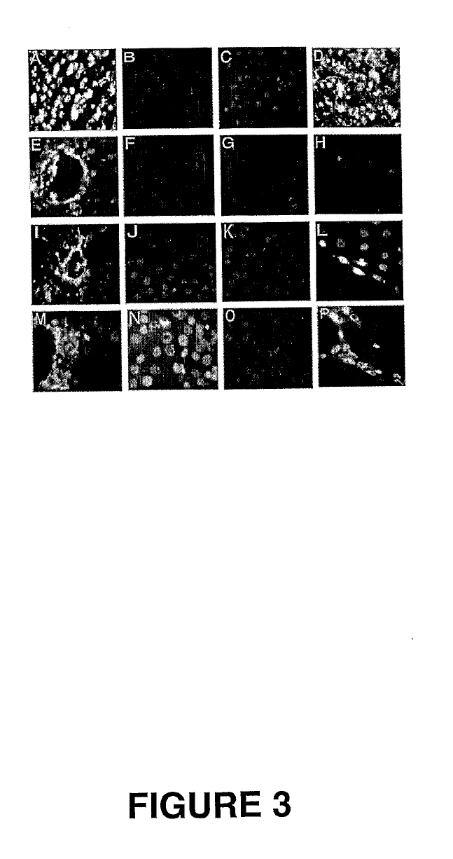

Several differences were also observed in the expression of cell surface markers between Thy1.1 positive and Thy1.1 negative cell populations. Both cell lines expressed CD147, CD44 and CD49f. Both were CD71 low and did not express the haematopoetic markers CD31, CD34, CD45 and c-kit. In contrast to the Thy1.1 positive sorted cell population, the Thy1.1 negative population was positive for CD24 (FIGS. 2a and 2b). The sub populations were also assessed by immunocytochemistry with hepatic, biliary and mesenchymal markers albumin, vimentin, CK7 and CK19 (FIG. 3).

Both Thy1.1 sorted populations were negative for albumin, CK 7 and vimentin (FIG. 3—panels B,C,F,G,J and K). The Thy1.1 positive population was also negative for CK 19 (FIG. 3, panel O), whereas the Thy1.1 negative cells were weakly positive (FIG. 3, panel N). Both populations were positive for c-Met and nestin by RT PCR (data not shown).

In summary, the Thy1.1 negative cell population expresses the following cell surface marker profile:

| | |
|---|---|
| Thy1.1 (CD90) | Negative |
| Pdx-1 | Positive |
| CD49f | ~95%+ |
| CD24 | ~80%+ |
| CD147 | ~80%+ |
| CD45 | Negative |
| CD44 | ~60%+ |
| CD71 | low |
| CD31 | Negative |
| c-Kit | Negative |
| ck7 | Negative |
| CK19 | Weak Positive |
| c-Met | Positive |

The Thy1.1 positive cells express the following cell surface marker profile:

| | |
|---|---|
| Thy1.1 (CD90) | Positive |
| Pdx-1 | Positive |
| CD49f | ~95%+ |
| CD24 | Negative |
| CD147 | ~90%+ |
| CD45 | Negative |
| CD44 | ~85%+ |
| CD71 | low |
| CD31 | Negative |
| C-KIT | Negative |
| CK19 | Negative |
| c-Met | Positive |
| Nestin | Positive |

### Differentiation Capacity of Thy1.1 Positive and Thy1.1 Negative Populations.

### Pancreatic Differentiation

Thy1.1 positive and Thy1.1 negative populations exhibited markedly different morphological changes in pancreatic differentiation media. The Thy1.1 positive population, initially fibroblast-like in morphology, formed matted cell clusters by days 14-21, and formed into islet-like spherical clusters by day 28, which eventually detached from the parent cell layer (FIG. 1, panels D,H,I). In contrast, the Thy-1.1 negative cells remained in a monolayer with a small epithelial like morphology, with no development of three dimensional structures (FIG. 1, panels A and B).

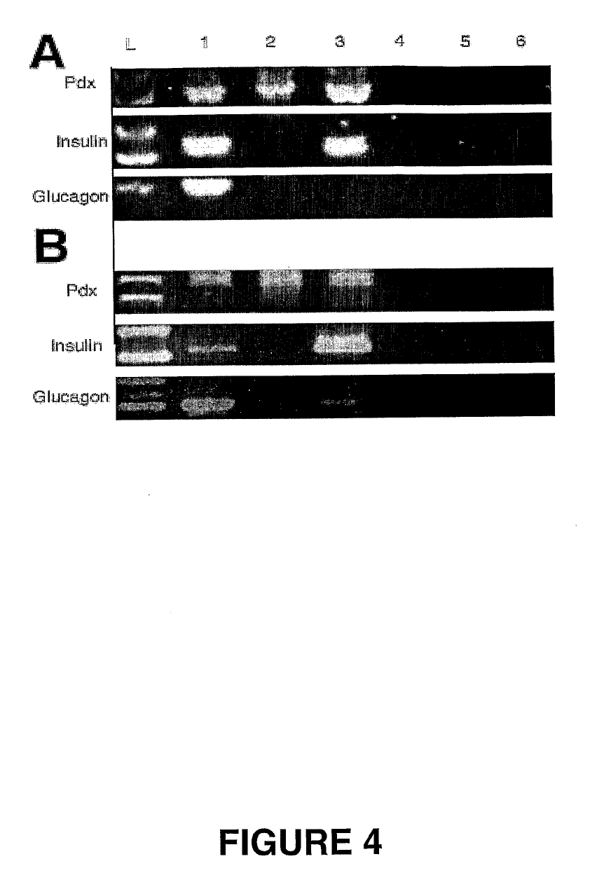

RT-PCR analysis of undifferentiated Thy1.1 positive cells demonstrated positive Pdx-1 expression, but no expression of insulin or glucagon (FIG. 4-B). Differentiated cell clusters all positive for the transcriptional expression of all three markers (FIG. 4 panel B).

Thy1.1 negative cells, however, expressed Pdx-1 when grown in either maintenance, or differentiation media. Notably, when grown in differentiation medium, despite showing no morphological changes, insulin transcription was detected in the Thy1.1 negative population. Glucagon was not expressed in the undifferentiated Thy-1.1 negative cells nor was it induced in vitro after differentiation (FIG. 4 panel A).

### Hepatic Differentiation

Thy-1.1 positive and negative PDPCs were culture in serum free, FGF4 containing media to assess hepatic potential. Thy-1.1 positive cells demonstrated a morphological change from fibroblastoid like to epithelial/cuboidal morphology. Furthermore by day 28 luminal structures were evident throughout the culture with flattened epithelium. Occasional three dimensional islet like structure similar to those seen in the pancreatic differentiation plates were also observed in the hepatic differentiation plates. The Thy-1.1 negative population remained in a monolayer with no evidence of three dimensional structures or of lumen like structures and no marked change in morphology (FIG. 1A,C).

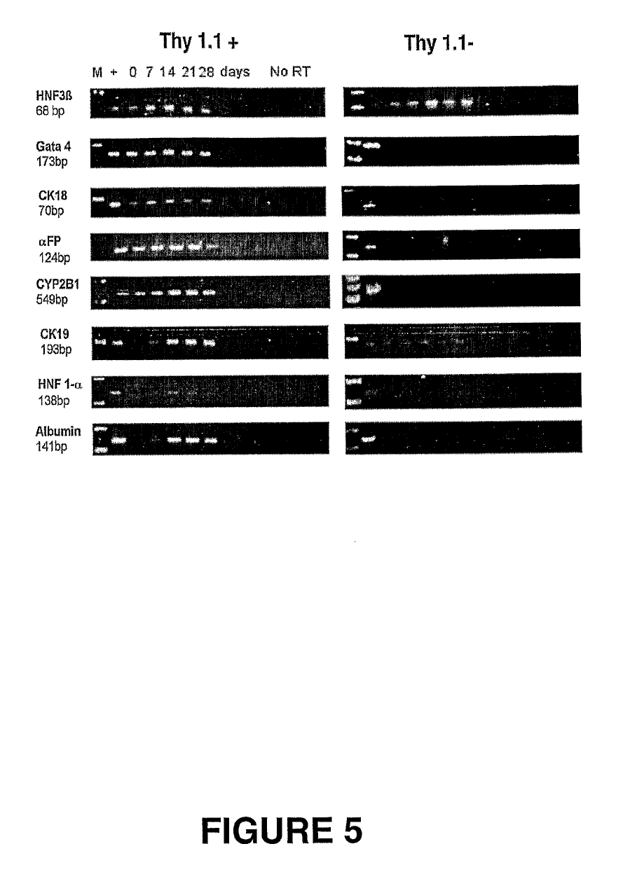

The inventor further examined the differentiation of the two populations by RT-PCR over a 28 day period. RT-PCR was performed for endodermal specific genes HNF3 beta and GATA 4, early liver maker alpha feto protein and CK18, mature liver markers HNF1alpha, albumin and the Cytochrome P450 enzyme CYP2B1. Undifferentiated Thy1.1 negative cells expressed HNF3-Beta and CK19 by RT-PCR, but did not express albumin, CK18, HNF1alpha, CY2B1, Gata4 or alpha fetoprotein (FIG. 5, panel B) None of the other early, or mature liver markers, or CY2B 1 were induced in the Thy1.1 negative population. Interestingly, undifferentiated Thy1.1 positive PDPCs expressed the early endodermal markers HNF3-Beta, GATA 4 and alpha fetoprotein, but did not express later markers of hepatocyte differentiation, HNF1-alpha and albumin, until day 14 of hepatic differentiation (FIG. 5 panel A). CK 19, normally expressed by biliary cells, was also induced during days 14-28 consistent with the appearance of the lumenal structures in culture. The induction of albumin expression was confirmed by immunocytochemistry. Undifferentiated cells, stained negatively for albumin content (FIG. 3, Panel A), while day 14 differentiated cells were strongly positive for albumin staining (FIG. 3, Panel D). Interestingly, differentiated cells at the day 10 and 14 time points, stained negatively for the biliary markers CK19 and CK7 (FIG. 3, Panels N and G), but did stain positively for vimentin in the lumen like structures only (FIG. 3, Panel L). CK 18 was also expressed in undifferentiated Thy1.1 positive cells and throughout the 28 day differentiation period. CYP2B 1 was present in undifferentiated Thy1.1 positive cells and throughout the differentiation period.

Undifferentiated Thy1.1 negative cells were negative for CK7, vimentin and albumin expression and weakly positive for CK19 by immunocytochemistry. The Thy1.1 positive population was negative for CK 19

### Periodic Acid Schiff (PAS) Staining for Glycogen Storage

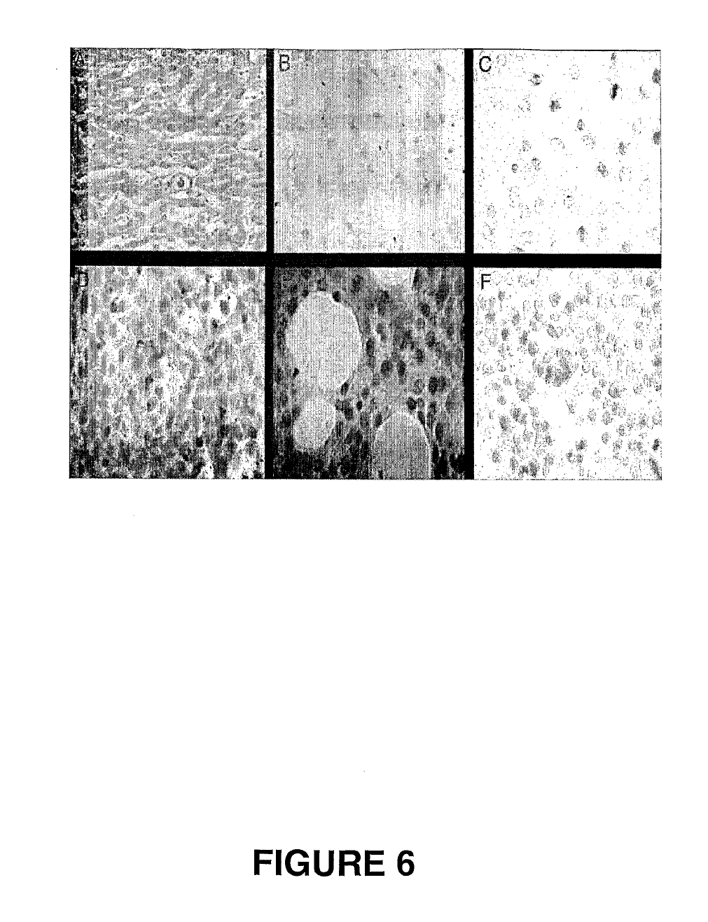

The presence of stored glycogen, as determined by PAS staining, was not observed in Thy-1.1 positive or negative PDPCs nor in day 21 differentiated Thy-1.1 negative cells. However positive staining with PAS indicative of glycogen storage was observed in the Thy1.1 positive differentiated cells by day 21 (FIG. 6).

### Examination of Cell Subpopulations in an Animal Model of Diabetes

A cell subpopulation described herein (e.g., a Thy1.1 positive or Thy1.1 negative cell population) can be employed in an animal model of disease such as diabetes. In one embodiment, a subpopulation of cells is used in a rodent concordant xenograft model of streptozotocin (STZ) induced diabetes. C57BL/6 mice are made diabetic by injection of STZ on day 0, while 750,000 cells (e.g., Thy1.1 positive cells) are injected into the tail vein of treated animals on day 3. Control animals are given an injection of saline or an equivalent number of C57BL/6 bone marrow cells. Blood glucose is monitored every 3 days. Stabilization of blood glucose and/or increased survival relative to controls indicate that the administered cells give rise to insulin production in the animals.

### Discussion

Provided herein are details on the in vitro culture, selection and characterisation of Thy1.1 positive and Thy1.1 negative PDPC sub-populations. Furthermore, there is disclosed details as to their potency with respect to differentiation to pancreatic and hepatic lineages and the provision of a cell population sorted using the marker Thy1.1, which displays lineage bipotentiality in vitro.

Thy1.1 is a cell surface protein whose function is not clearly understood. However, it has been suggested to be involved in cellular recognition (Gunter et al., 1984; Williams, 1985), cellular adhesion (He et al., 1991; Hueber et al., 1992) and signal transduction (Kroczek et al., 1986). Thy1.1 expression observed in various stem cell populations, notably the oval cell population in adult rat liver, has led to the supposition that Thy1.1 may allow cells to recognize and adhere to stromal tissue, potentially as repair cells after injury. (Masson et al., 2006; Petersen et al., 1998; Terrace et al., 2007). Thy1.1 is also expressed on stem cells of the fetal liver, umbilical cord blood and mesenchymal stein cells in humans, mouse and rat. The present findings of greater in vitro potency within the Thy1.1 positive population would be consistent with these observations. They also demonstrate a method of isolation and purification with which to enable further use of such cells.

Previous studies have demonstrated hepatic differentiation of a number of different cell types, including bone marrow derived MSCs, MAPCs, endometrial and pancreatic derived MSCs (Jiang et al., 2002; Meng et al., 2007; Schwartz et al., 2002; Seeberger et al., 2006). Thy1.1 positive subpopulations of PDPCs share the morphological phenotype and express a number of cell surface markers with these populations, including CD44+, CD24−, CD45−, CD31− and CD34. In contrast to this however, Thy1.1 positive PDPCs appear to be a distinct cell type, expressing GATA4, HNF3-beta and alpha feto protein, which have not been described as expressed for any of these other cell types.

HNF3-beta is a marker of definitive endoderm believed to play an important role in endoderm competency (Gualdi et al 1996) while GATA4 is a transcription factor required for ventral foregut endoderm development and for early liver gene expression (Gualdi et al., 1996; Rossi et al., 2001). HNF3-beta has been demonstrated to direct nucleosome positioning within the context of the albumin enhancer (McPherson et al., 1996; Cirillo and Zaret, 1999) with the subsequent facilitation of binding of GATA4 to the albumin enhancer. Both GATA4−/− and HNF3-beta−/− embryos show defects in foregut morphogenesis (Duncan et al., 1997). Therefore the expression of HNF3-beta and GATA4 in undifferentiated PDPCs and the subsequent FGF stimulated induction of expression of liver specific genes such as albumin and HNF1-alpha, is consistent with the proposal that HNF 3-beta and GATA 4 co-operate to control the potential of these cells to commit to a hepatic fate. Moreover, the presence of PAS staining in the Thy1.1 positive population after 21 days of differentiation, demonstrated a functional characteristic of more mature hepatocytes, which is consistent with the expression of HNF1-alpha. HNF1-alpha is known to bind to genes whose products are related to mature hepatic functions, including carbohydrate storage and synthesis and lipid metabolism (Odom et al., 2004).

Undifferentiated Thy1.1 positive PDPCs express AFP. This observation is consistent with reports describing AFP expression in Nestin positive islet derived progenitor cells and low level AFP and TTR expression, prior to hepatogenesis in the early ventral foregut endoderm. This expression is subsequently lost in endoderm isolated from cardiac mesodermal signalling (Gualdi et al., 1996; Jung et al., 1999; Zulewski et al., 2001). It has also been suggested that this is a feature of the default pancreatic fate of ventral foregut endoderm, (Deusch et als). Expression of AFP in the Thy1.1 positive PDPC population, which demonstrates capacity to both pancreatic and hepatic lineages, would not be inconsistent with this finding. (Deutsch et al., 2001)

Significantly, the undifferentiated Thy1.1 negative population, while expressing HNF3beta, did not express GATA 4, or alpha feto-protein, nor were they induced during the differentiation experiment. No evidence of hepatic competency was observed in the Thy1.1 negative population. This is congruent with Pdx-1 expression and the absence of Gata4 expression within this undifferentiated population. Vimentin was not expressed in either undifferentiated Thy1.1 positive or negative populations but was expressed in the cells forming the ductal-like structures during hepatic differentiation. Vimentin is considered to represent a mesenchymal marker. However, Masson et al observed coexpression of Thy1.1 and vimentin in portal structures, as well as demonstrating vimentin expression in epithelial cells within tissue sections and in culture of fetal liver epithelial cells. (Masson et al., 2006)

The data pertaining to pancreatic differentiation are intriguing. No morphological evidence of islet like clusters was observed in the Thy1.1 negative population. In contrast, Thy1.1 positive PDPCs could readily be induced to a pancreatic lineage with characteristic morphological changes resulting in three dimensional islet like structures and the transcriptional expression of PDX-1, insulin and glucagon.

The detection of Pdx-1 transcriptional expression in both populations indicates their potential to become insulin producing cells. Notably, however, Thy1.1 negative cells when grown in differentiation medium, despite showing no morphological changes, expressed insulin. Glucagon was not expressed in the undifferentiated Thy-1.1 negative cells, nor was it induced in vitro after differentiation (FIG. 4 panel).

Various different candidate populations of pancreatic progenitor/stem cell have been described previously, including islet progenitor cells expressing nestin or other neuronal stem cell markers, (Abraham et al., 2004; Cornelius et al., 1997; Lechner et al., 2002; Ramiya et al., 2000). Another population have been shown to express PDX-1, a known marker for insulin producing cells and these cells can stimulate both ductal and endocrine differentiation in vitro under appropriate conditions (Bonner-Weir et al., 2000; Otonkoski et al., 1993). Moreover, there is evidence that pancreatic ductal epithelial cells have the potential to dedifferentiate to a progenitor cell capable of proliferation and formation of new islets and acini (Bonner-Weir et al., 2004) and most recently, CK19+ Non-Endocrine Pancreatic Epithelial cells (NEPCs) were reported to be partially induced to differentiate into insulin producing cells in vivo, when in the presence of fetal pancreatic tissue (Hao et al., 2006).

The precise physiological role played by these cells has been questioned. Dor et al. have challenged the view that neogenesis from ductal or progenitor cells occurs, instead arguing that beta cell replication, rather than new islet generation is the predominant mechanism by which pancreatic endocrine tissue regenerates after near-total pancreatectomy (Dor et al., 2004) although this interpretation still remains controversial (Bonner-Weir and Weir, 2005). The present findings are consistent with a role for a non beta cell that can produce insulin as potentially facilitating such a process. It is clear that these cells offer an alternative insulin producing cell source for transplantation therapies.

The inventor observed a time course of both morphologic and gene expression changes indicative of hepatic lineage differentiation by use of a serum free FGF-4 containing differentiation protocol. The potential bipotentiality of embryonic ventral endoderm for pancreas and liver differentiation has been investigated in explant experiments where ventral endoderm differentiated to hepatic lineage by proximity to the cardiac mesoderm. The absence of inductive factors, such as FGF-1, FGF-2 and FGF-4, secreted by cardiac mesoderm allow the default pancreatic pathway of ventral endoderm to continue (Deutsch et al., 2001) and general FGF signalling antagonist inhibits heptogenesis in vitro (Jung et al., 1999) and FGF-4 (Zhu et al., 1999). The present data are entirely congruent with this concept.

The inventor has demonstrated isolation and characterisation of PDPCs, which in vitro, demonstrate potency and transcriptional responses to signalling consistent with a population of bipotential endodermal progenitors. Previously, administration of unsorted PDPC populations in a murine streptozocin induced diabetes model have demonstrated differentiation and production of rat insulin with concurrent stimulation of mouse pancreatic regeneration (Shiels 2005 and WO 2006/120476, both incorporated herein by reference).

### REFERENCE LIST

1. Abraham, E. J., S. Kodama, J. C. Lin, M. Ubeda, D. L. Faustman and J. F. Habener: Human pancreatic islet-derived progenitor cell engraftment in immunocompetent mice. Am. J. Pathol. Dev. 164, 817-830 (2004).
2. Bonner-Weir, S., E. Toschi, A. Inada, P. Reitz, S. Fonseca, T. Aye and A. Shanna: The pancreatic ductal epithelium serves as a potential pool of progenitor cells. Paediatric Diabetes Dev. 5, 16-22 (2004).
3. Bonner-Weir, S., M. Taneja, G. C. Weir, K. Tatarkiewicz, K. H. Song, A. Sharma and J. J. O'Neil: In vitro cultivation of human islets from expanded ductal tissue Proc. Natl. Acad. Sci. U.S.A Dev. 97, 7999-8004 (2000).
4. Bonner-Weir, S. and G. C. Weir: New sources of pancreatic beta-cells. Nat. Biotechnol. Dev. 23, 857-861 (2005).
5. Cirillo, L. A. and K. S. Zaret: An early developmental transcription factor complex that is more stable on nucleosome core particles than on free DNA. Mol. Cell Dev. 4, 961-969 (1999).
6. Cornelius, J. G., V. Tchernev, K. J. Kao and A. B. Peck: In vitro-generation of islets in long-term cultures of pluripotent stem cells from adult mouse pancreas. Horm. Metab Res. Dev. 29, 271-277 (1997).
7. Dabeva, M. D., S. G. Hwang, S. R. Vasa, E. Hurston, P. M. Novikoff, D. C. Hixson, S. Gupta and D. A. Shafritz: Differentiation of pancreatic epithelial progenitor cells into hepatocytes following transplantation into rat liver. Proc. Natl. Acad. Sci. U.S.A Dev. 94, 7356-7361 (1997).
8. Deutsch, G., J. Jung, M. Zheng, J. Lora and K. S. Zaret: A bipotential precursor population for pancreas and liver within the embryonic endoderm. Development Dev. 128, 871-881 (2001).
9. Dor, Y., J. Brown, O. I. Martinez and D. A. Melton: Adult pancreatic beta-cells are formed by self-duplication rather than stem-cell differentiation 1. Nature Dev. 429, 41-46 (2004).
10. Duncan, S. A., A. Nagy and W. Chan: Murine gastrulation requires HNF-4 regulated gene expression in the visceral endoderm: tetraploid rescue of Hnf-4(−/−) embryos. Development Dev. 124, 279-287 (1997).
11. Gualdi, R., P. Bossard, M. Zheng, Y. Hamada, J. R. Coleman and K. S. Zaret: Hepatic specification of the gut endoderm in vitro: cell signaling and transcriptional control. Genes Dev. Dev. 10, 1670-1682 (1996).
12. Gunter, K. C., T. R. Malek and E. M. Shevach: T cell-activating properties of an anti-Thy-1 monoclonal antibody. Possible analogy to OKT3/Leu-4. J. Exp. Med. Dev. 159, 716-730 (1984).
13. Hao, E., B. Tyrberg, P. kin-Ansari, J. key, I. ron, E. nosov, M. rcova, M. rcola and F. vine: Beta-cell differentiation from nonendocrine epithelial cells of the adult human pancreas. Nat. Med. Dev. 12, 310-316 (2006).
14. He, H. T., P. Naquet, D. Caillol and M. Pierres: Thy-1 supports adhesion of mouse thymocytes to thymic epithelial cells through a Ca2(+)-independent mechanism. J. Exp. Med. Dev. 173, 515-518 (1991).
15. Hueber, A. O., M. Pierres and H. T. He: Sulfated glycans directly interact with mouse Thy-1 and negatively regulate Thy-1-mediated adhesion of thymocytes to thymic epithelial cells. J. Immunol. Dev. 148, 3692-3699 (1992).
16. Jiang, Y., B. N. Jahagirdar, R. L. Reinhardt, R. E. Schwartz, C. D. Keene, X. R. Ortiz-Gonzalez, M. Reyes, T. Lenvik, T. Lund, M. Blackstad, J. Du, S. Aldrich, A. Lisberg, W. C. Low, D. A. Largaespada and C. M. Verfaillie: Pluripotency of mesenchymal stem cells derived from adult marrow. Nature Dev. 418, 41-49 (2002).
17. Jung, J., M. Zheng, M. Goldfarb and K. S. Zaret: initiation of mammalian liver development from endoderm by fibroblast growth factors. Science Dev. 284, 1998-2003 (1999).
18. Krakowski, M. L., M. R. Kritzik, E. M. Jones, T. Krahl, J. Lee, M. Arnush, D. Gu, B. Mroczkowski and N. Sarvetnick: Transgenic expression of epidermal growth factor and keratinocyte growth factor in beta-cells results in substantial morphological changes. J. Endocrinol. Dev. 162, 167-175 (1999a).
19. Krakowski, M. L., M. R. Kritzik, E. M. Jones, T. Krahl, J. Lee, M. Arnush, D. Gu and N. Sarvetnick: Pancreatic expression of keratinocyte growth factor leads to differentiation of islet hepatocytes and proliferation of duct cells. Am. J. Pathol. Dev. 154, 683-691 (1999b).
20. Kroczek, R. A., K. C. Gunter, B. Seligmann and E. M. Shevach: Induction of T cell activation by monoclonal anti-Thy-1 antibodies. J. Immunol. Dev. 136, 4379-4384 (1986).
21. Lechner, A., C. A. Leech, E. J. Abraham, A. L. Nolan and J. F. Habener: Nestin-positive progenitor cells derived from adult human pancreatic islets of Langerhans contain side population (SP) cells defined by expression of the ABCG2 (BCRP1) ATP-binding cassette transporter. Biochem. Biophys. Res. Commun. Dev. 293, 670-674 (2002).
22. Masson, N. M., I. S. Currie, J. D. Terrace, O. J. Garden, R. W. Parks and J. A. Ross: Hepatic progenitor cells in human fetal liver express the oval cell marker Thy-1. Am. J. Physiol Gastrointest. Liver Physiol Dev. 291, G45-G54 (2006).
23. McPherson, C. E., R. Horowitz, C. L. Woodcock, C. Jiang and K. S. Zaret: Nucleosome positioning properties of the albumin transcriptional enhancer. Nucleic Acids Res. Dev. 24, 397-404 (1996).
24. Meng, X., T. E. Ichim, J. Zhong, A. Rogers, Z. Yin, J. Jackson, H. Wang, W. Ge, V. Bogin, K. W. Chan, B. Thebaud and N. H. Riordan: Endometrial regenerative cells: A novel stem cell population. J. Transl. Med. Dev. 5, 57 (2007).
25. Odom, D. T., N. Zizlsperger, D. B. Gordon, G. W. Bell, N. J. Rinaldi, H. L. Murray, T. L. Volkert, J. Schreiber, P. A. Rolfe, D. K. Gifford, E. Fraenkel, G. I. Bell and R. A. Young: Control of pancreas and liver gene expression by HNF transcription factors. Science Dev. 303, 1378-1381 (2004).
26. Otonkoski, T., G. M. Beattie, M. I. Mally, C. Ricordi and A. Hayek: Nicotinamide is a potent inducer of endocrine differentiation in cultured human fetal pancreatic cells J. Clin. Invest Dev. 92, 1459-1466 (1993).
27. Petersen, B. E., J. P. Goff, J. S. Greenberger and G. K. Michalopoulos: Hepatic oval cells express the hematopoietic stem cell marker Thy-1 in the rat. Hepatology Dev. 27, 433-445 (1998).
28. Petersen, B. E., B. Grossbard, H. Hatch, L. Pi, J. Deng and E. W. Scott: Mouse A6-positive hepatic oval cells also express several hematopoietic stem cell markers. Hepatology Dev. 37, 632-640 (2003).
29. Ramiya, V. K., M. Maraist, K. E. Arfors, D. A. Schatz, A. B. Peck and J. G. Cornelius: Reversal of insulin-dependent diabetes using islets generated in vitro from pancreatic stem cells. Nat. Med. Dev. 6, 278-282 (2000).
30. Rao, M. S, and J. K. Reddy: Hepatic transdifferentiation in the pancreas. Semin. Cell Biol. Dev. 6, 151-156 (1995).
31. Rao, M. S., V. Subbarao and J. K. Reddy: Induction of hepatocytes in the pancreas of copper-depleted rats following copper repletion. Cell Differ. Dev. 18, 109-117 (1986).
32. Rossi, J. M., N. R. Dunn, B. L. Hogan and K. S. Zaret: Distinct mesodermal signals, including BMPs from the septum transversum mesenchyme, are required in combination for hepatogenesis from the endoderm. Genes Dev. Dev. 15, 1998-2009 (2001).
33. Schwartz, R. E., M. Reyes, L. Koodie, Y. Jiang, M. Blackstad, T. Lund, T. Lenvik, S. Johnson, W. S. Hu and C. M. Verfaillie: Multipotent adult progenitor cells from bone marrow differentiate into functional hepatocyte-like cells. J. Clin. Invest Dev. 109, 1291-1302 (2002).
34. Seeberger, K. L., J. M. Dufour, A. M. Shapiro, J. R. Lakey, R. V. Rajotte and G. S. Korbutt: Expansion of mesenchymal stem cells from human pancreatic ductal epithelium. Lab Invest Dev. 86, 141-153 (2006).
35. Shiels P G Adult stem cells mitigate the effects of STZ diabetes. Horizons in Medicine 17:241-249 Royal College of Physicians (D Haskard Ed)
36. Terrace, J. D., I. S. Currie, D. C. Hay, N. M. Masson, R. A. Anderson, S. J. Forbes, R. W. Parks and J. A. Ross: Progenitor cell characterization and location in the developing human liver. Stem Cells Dev. Dev. 16, 771-778 (2007).
37. Wang, X., M. Al Dhalimy, E. Lagasse, M. Finegold and M. Grompe: Liver repopulation and correction of metabolic liver disease by transplanted adult mouse pancreatic cells. Am. J. Pathol. Dev. 158, 571-579 (2001).
38. Williams, A. F.: Immunoglobulin-related domains for cell surface recognition. Nature Dev. 314, 579-580 (1985).
39. Zhu, X., J. Sasse and J. Lough: Evidence that FGF receptor signaling is necessary for endoderm-regulated development of precardiac mesoderm. Mech. Ageing Dev. Dev. 108, 77-85 (1999).
40. Zulewski, H., E. J. Abraham, M. J. Gerlach, P. B. Daniel, W. Moritz, B. Muller, M. Vallejo, M. K. Thomas and J. F. Habener: Multipotential nestin-positive stem cells isolated from adult pancreatic islets differentiate ex vivo into pancreatic endocrine, exocrine, and hepatic phenotypes. Diabetes Dev. 50, 521-533 (2001).

1. An isolated cell population originating from adult tissue, wherein said cells are capable of growth in a matrigel free culture system in the presence of serum; characterised in that the cells are Pdx-1 positive.

2. An isolated cell population according to claim 1 wherein said cells are Nestin negative.

3. An isolated cell population according to claim 1 wherein said cells are Nestin positive.

4. An isolated cell population according to claim 1 wherein the adult tissue in pancreatic, bone marrow, heart, breast, liver or kidney tissue.

5. An isolated cell population according to claim 3 wherein the adult tissue is human.

6. (canceled)

7. An isolated cell population according to claim 6 wherein the cells are derived from cells deposited under accession number Q6203 at ECACC on 12 May 2005.

8. An isolated cell population according to claim 1 wherein the cells are positive for the cell surface marker Thy 1.1.

9. An isolated cell population according to claim 1 wherein the cells are negative for the cell surface marker Thy1.1.

10. A pharmaceutical composition comprising an isolated cell population according to claim 1 and a pharmaceutically acceptable carrier.

11. A method of isolating a bipotent stem cell population from adult mammalian tissue, said method comprising: culturing said adult mammalian tissue; isolating emergent cell population monolayer; and further isolating those cells which are positive for the cell surface marker Thy 1.1 thereby providing a bipotent stem cell population.

12. A method according to claim 11 wherein said isolated cells are also positive for one or more cell surface markers selected from the group consisting of CD 147, CD44, CD49F, C-Met and Nestin.

13-14. (canceled)

15. A method according to claim 11 wherein the tissue is cultured in CMRL-1066 medium (sigma-C-0422).

16. A method according to claim 11 wherein the tissue is from adult pancreatic ducts comprising whole ducts.

17. A method according to claim 16 wherein the duct tissue is minced to aid in the culturing step.

18. A method of isolating a bipotent stem cell population, said method comprising obtaining a population of cells as deposited under accession number Q6203 at ECACC on 12 May 2005; and isolating a sub-population of cells which are positive for the cell surface marker Thy 1.1.

19. An adult cell population obtainable by a method according to claim 11.

20. A method of treating a disease state associated with cell degeneration or age related tissue change, said method comprising the steps of administering a cell population according to claim 1 to a patient having said disease.

21. A method according to claim 20 wherein the cells are administered intravenously to said patient, or are transplanted to the disease site or site of age related degeneration.

22. (canceled)

23. A method according to claim 20 wherein the disease is associated with degeneration of pancreatic cells, neuronal cells, cardiovascular cells, epithelial cells, liver cells, muscle cells, retinal cells, hair follicle or kidney cells.

24. A method according to claim 23 wherein the disease is diabetes (Type I or II), Parkinson's disease, Alzheimer's disease, kidney disease, eye disease, liver disease or cardiovascular disease.

25. A method according to claim 20 wherein the donor of the cells and the patient are the same species.

26. (canceled)

27. A method of isolating an adult stem cell population according to claim 9 from adult mammalian tissue, said method comprising culturing said adult mammalian tissue; isolating emergent cell population monolayer; and further isolating those cells which are negative for the cell surface marker Thy 1.1.

28. A method according to claim 27 wherein said isolated cells are positive for one or more cell surface markers selected from the group consisting of CD 147, CD49f, CD44, CK19, C-Met and Nestin.

29-30. (canceled)

31. A method of isolating an adult stem cell population according to claim 9, said method comprising obtaining a population of cells as deposited under accession number Q6203 at ECACC on 12 May 2005; and isolating a sub-population of cells which are negative for the cell surface marker Thy1.1.

32. An isolated adult stem cell population according to claim 8 for use in a method of treatment.

33-36. (canceled)

37. An adult cell population obtainable by a method according to claim 18.

38. A method of treating a disease state associated with cell degeneration or age related tissue change, said method comprising the steps of administering a pharmaceutical composition according to claim 10 to a patient having said disease.


SEQUENCE LISTING (25 sequences)

SEQ ID NO: 1 - DNA, 21 bp, Artificial Sequence
  Synthetic sequence Oligonucleotide primer
cggccacacagctctacaagg

SEQ ID NO: 2 - DNA, 21 bp, Artificial Sequence
  Synthetic sequence Oligonucleotide primer
ctccggttctgctgcgtatgc

SEQ ID NO: 3 - DNA, 20 bp, Artificial Sequence
  Synthetic sequence Oligonucleotide primer
ttccaggcccccagtctcgg

SEQ ID NO: 4 - DNA, 20 bp, Artificial Sequence
  Synthetic sequence Oligonucleotide primer
atggccctgtggatccgctt

SEQ ID NO: 5 - DNA, 20 bp, Artificial Sequence
  Synthetic sequence Oligonucleotide primer
tgccaaggtctgaaggtcac

SEQ ID NO: 6 - DNA, 21 bp, Artificial Sequence
  Synthetic sequence Oligonucleotide primer
cctgctcatcctctgggagcc

SEQ ID NO: 7 - DNA, 18 bp, Artificial Sequence
  Synthetic sequence Oligonucleotide primer
gaccgtttacgtggctgg

SEQ ID NO: 8 - DNA, 24 bp, Artificial Sequence
  Synthetic sequence Oligonucleotide primer
cggttcctcttggtgttcatcaag

SEQ ID NO: 9 - DNA, 21 bp, Artificial Sequence
  Synthetic sequence Oligonucleotide primer
acaaggcagctggcagcatgc

SEQ ID NO: 10 - DNA, 24 bp, Artificial Sequence
  Synthetic sequence Oligonucleotide primer
ctgggagtgtgcagatatcagagt

SEQ ID NO: 11 - DNA, 24 bp, Artificial Sequence
  Synthetic sequence Oligonucleotide primer
gagaaggtcaccaagtgctgtagt

SEQ ID NO: 12 - DNA, 24 bp, Artificial Sequence
  Synthetic sequence Oligonucleotide primer
cctactcgtacatctcgctcatca

SEQ ID NO: 13 - DNA, 19 bp, Artificial Sequence
  Synthetic sequence Oligonucleotide primer
cgctcagcgtcagcatctt

SEQ ID NO: 14 - DNA, 21 bp, Artificial Sequence
  Synthetic sequence Oligonucleotide primer
agctgctcctccatcatcaga

SEQ ID NO: 15 - DNA, 29 bp, Artificial Sequence
  Synthetic sequence Oligonucleotide primer
tgttccaagcattaagttttctattctaa

SEQ ID NO: 16 - DNA, 20 bp, Artificial Sequence
  Synthetic sequence Oligonucleotide primer
catgcttgcagttgtgctag

SEQ ID NO: 17 - DNA, 20 bp, Artificial Sequence
  Synthetic sequence Oligonucleotide primer
attctctgctacggccagta

SEQ ID NO: 18 - DNA, 22 bp, Artificial Sequence
  Synthetic sequence Oligonucleotide primer
gtcctttcttcctcctggagat

SEQ ID NO: 19 - DNA, 22 bp, Artificial Sequence
  Synthetic sequence Oligonucleotide primer
ctgtcactgctgatttctctgg

SEQ ID NO: 20 - DNA, 21 bp, Artificial Sequence
  Synthetic sequence Oligonucleotide primer
gagttcttctctgggttcctg

SEQ ID NO: 21 - DNA, 20 bp, Artificial Sequence
  Synthetic sequence Oligonucleotide primer
actgtgggtcatggagagct

SEQ ID NO: 22 - DNA, 20 bp, Artificial Sequence
  Synthetic sequence Oligonucleotide primer
agtaacgtgcgtgctgacac

SEQ ID NO: 23 - DNA, 20 bp, Artificial Sequence
  Synthetic sequence Oligonucleotide primer
agtcgcactggtagcaaggt

SEQ ID NO: 24 - DNA, 21 bp, Artificial Sequence
  Synthetic sequence Oligonucleotide primer
ggacctcagcaagatcatggc

SEQ ID NO: 25 - DNA, 21 bp, Artificial Sequence
  Synthetic sequence Oligonucleotide primer
ccacgatcttacgggtagttg